(19) United States
(12) Patent Application Publication
(10) Pub. No.: US 2007/0273867 A1
(43) Pub. Date: Nov. 29, 2007

(54) Ir-Atr-Based Process for Analyzing Very Small Amounts of Sample in the Nanoliter Range

(76) Inventors: Edgar Diessel, Koln (DE); Stefan Willmann, Dusseldorf (DE); Peter Kamphaus, Leverkusen (DE); Frank Lison, Gauting (DE)

Correspondence Address: NIXON PEABODY LLP, 161 N. CLARK STREET, 48TH FLOOR, CHICAGO, IL, 60601-3213, US

(21) Appl. No.: 10/561,130

(22) PCT Filed: Jun. 23, 2004
(86) PCT No.: PCT/IB04/02135

§ 371 Date: Jun. 22, 2007

(30) Foreign Application Priority Data
Jun. 27, 2003 (DE) 10328998.4

(51) Int. Cl. G01N 1/28; G01N 33/49; G01N 33/493

(52) U.S. Cl. 356/36

(57) ABSTRACT

A process and an are described, which enable the analysis of very small amounts of sample (2) by infrared spectroscopy method. The invention relates in particular to a method which includes the following steps: aspirating the liquid sample (2) into a metering apparatus (1), metering the sample (2) onto the optical element (4) of an ATR apparatus, drying the sample (6), carrying out an ATR-IR spectroscopy measurement, forecasting the concentration of the analytes to be detected using a model-based calibration procedure.

The invention relates to a method and apparatus for the infrared spectroscopy, in vitro analysis of very small amounts of samples, for example for the medical diagnosis of human body fluids, for example interstitial liquid or blood. Spectroscopic analyses may also be used for the monitoring and control of biotechnology processes in the pharmaceutical, agricultural and foods industry. Spectroscopy avoids the use of reactants and offers the possibility of simultaneous determination of many components in one sample. In particular, the invention relates to a combination of methods for mechanical metering and for total internal reflection (ATR) spectroscopy and to a corresponding apparatus for performing this method.

It is widely accepted that methods based on infrared (IR) have great potential for the reagent-free analysis of samples. A review of their use in clinical applications is given, for example, in H. M. Heise, Infrared and Raman Spectroscopy of Biological Materials, eds. H. Gremlich, B. Yan, Marcel Deckker (New York, Basle), 2001, p. 259-322. Near infrared spectroscopy (NIR), characterized by a wavenumber range of 4000 to 14 000 cm⁻¹, addresses the harmonics of vibration bands of the analytes to be detected. Fundamental vibration bands are analysed by middle infrared spectroscopy (MIR) which covers the wavelength range between 800 and 4000 cm⁻¹. While NIR is developed principally for the in vivo analysis of body fluids, MIR is preferred for in vitro analysis. This is connected with the higher spectral resolution. For example, the fingerprint region of glucose, a very important clinical analyte, is in the region between 900 and 1200 cm⁻¹. As a consequence of the high water absorption, MIR is less preferred for in vivo analysis. Although the horizon of the present invention is not restricted to MIR and thus also encompasses NIR, MIR constitutes the preferred method for the implementation of the invention. In vitro MIR analysis of liquid samples interferes with the high water absorption. This absorption is markedly reduced by drying the sample. For clinical analysis, for example in the point-of-care field, small sample volumes in the μl and sub-μl range are to be analysed. This requirement is satisfied by a diffuse reflection method for analysing a 1 μl blood sample which is described by G. H. Werner et al., SPIE Vol. 3257, p. 91-100. Microdialysis samples of volume 1 μl were tested using a fibre optic-based ATR apparatus by H. M. Heise et al., Spectrochimica Acta Part B 57 (2002), p. 1649-1663. Even though signal-to-noise ratios would allow the analysis of samples having small volumes down to the submicrolitre range, manual sample application remains a significant source of error. A mechanical metering apparatus for μl volumes for a reflection-based method is described in U.S. Pat. No. 5,334,837. Even volumes of 0.5 nl have been metered by a piezo-driven microdropper for transmission spectroscopy (M. Haberkorn et al., Applied Spectroscopy 56 (2002), p. 902-908). It is not a simple task to comment either transmission or reflection apparatus and a metering apparatus together in a fixed manner in one instrument. Therefore, the sample is prepared here at a certain point and subsequently transferred to the sample stage for the spectroscopic analysis. Both for transmission and reflection methods, the sample volume has to be set carefully. Lower demands are needed for ATR-based methods which are in particular surface-sensitive.

In summary: there exist IR technologies for micrometering apparatus for the preparation of μl and sub-μl samples which are subsequently investigated by transmission or reflection methods.

The object on which the invention is based is to provide a method and an apparatus which enable the IR analysis of very small amounts of sample, which is characterized by simpler handling and reduced demands for the precision of the sample volume to be set.

The achievement of the object of the invention consists in a method and an apparatus which allow the spectroscopic analysis of very small amounts of sample. The invention relates in particular to a method which includes the following steps: aspirating the liquid sample into a metering apparatus, metering the sample onto the optical element of an ATR apparatus, drying the sample, carrying out an ATR-IR spectroscopy measurement, forecasting the concentration of the analytes to be detected using a model-based calibration procedure. Preference is given to MIR spectroscopy. In a preferred embodiment, it is especially glucose in human body fluids that is detected. An apparatus is claimed which combines a metering apparatus with an ATR apparatus in one instrument.

The invention provides a process for analysing a liquid sample which comprises one or more analytes and includes the following steps: aspirating the sample into a metering apparatus, metering the sample onto the measurement surface of an ATR apparatus by means of the metering apparatus, measuring the ATR-IR spectrum, analysing the spectrum with a calibration procedure for concentration determination of the analytes to be detected, characterized in that the sample is dried on the measurement surface before the ATR-IR spectrum is measured.

The sample is preferably selected from the group of: interstitial fluid, blood, serum, plasma, urine, saliva, sweat or lacrimal fluid.

The analyte is preferably selected from the group of: glucose, high-density lipoproteins (HDL), low-density lipoproteins (LDL), cholesterol, triglycerides, albumin, total protein alone or in any combination, urea, uric acid, haemoglobin and/or creatinine.

The process is preferably carried out in such a way that the NIR spectrum is measured in the wavenumber range of 800 to 14 000 cm⁻¹, preferably in the wavenumber range of 900 to 1200 cm⁻¹.

In a preferred process, sample volumes of 0.2 to 1000 nl, preferably 0.5 to 500 nl, are used and analysed.

The sample may be metered onto the ATR apparatus in one drop.

However, in a preferred variant of the process, the sample is applied to the measurement surface of the ATR apparatus in several steps as a drop sequence, the drop sequence in each case consisting of one or more drops and the sample being dried between the drop sequences.

The sample is preferably dried by supplying heat, by passing over drying gas or evacuating the sample chamber.

The measurement surface is advantageously bounded by a frame on the optical element of the ATR apparatus.

In addition to the spectroscopic data, morphological data of the dried sample can be determined experimentally. Preference is therefore given to a process which is characterized in that, in addition to the IR spectrum, the morphology of the dried sample is determined, especially by imaging the sample or interferometry.

The morphological information consists, for example, of the surface area of the dried sample, in the volume or in a three-dimensional height profile of the dried sample.

The morphological information may be generated, for example, by measuring the distance from the surface of the dried film to a reference point by means of a focused laser beam.

The spectroscopic method to be used is preferably based on FTIR or based on the reflection of discrete wavelengths in the NIR and/or MIR region.

The spectroscopic method may be based on the reflection of a test beam which is emitted by a narrowband IR source.

Alternatively, the spectroscopic method may be based on a broadband IR emitter and one or more detectors which are selected either simultaneously or successively and are sensitive to different wavelength ranges, which is realised by detectors having broadband sensitivity in conjunction with wavelength-sensitive filters or by adjustable detectors having narrowband sensitivity.

The concentrations of one or more analytes may be derived from spectroscopic data, the appropriate concentration model being based on partial least squares (PLS) or neural networks.

The concentrations of one or more analytes may also be derived as described above from spectroscopic data and morphological data, the appropriate concentration model being based on PLS or neural networks.

The invention further provides an apparatus for performing the process according to the invention comprising at least a combination of a metering apparatus with an ATR spectrometer apparatus, optionally with dryer unit, characterized in that the metering apparatus meters drop volumes in the range from 0.2 to 1000 nl, preferably 0.5 to 500 nl.

The metering apparatus is preferably a piezo-driven dropper or a syringe-driven dropper.

The apparatus is more preferably mounted in an evacuable casing and equipped with additional means of evacuation.

The apparatus may preferably additionally be equipped with a blower and feed line connected thereto, and also one or more nozzles which are connected to the feed line and are directed towards the measurement surface of the optical element of the ATR apparatus.

The gas which dries the sample is more preferably preheated in such an arrangement.

The optical element of the ATR apparatus may also be heated by a separate IR source.

Drying of the sample is also enabled when the optical element of the ATR apparatus is mounted in a holder which can be heated by an electrical current.

The optical element of the ATR measurement apparatus is preferably a prism, a planar waveguide or a plate waveguide and is in each case manufactured from diamond, silicon, zinc selenide or germanium.

In a preferred variant of the apparatus, the optical element of the ATR measurement apparatus is an optical fibre manufactured from chalcogenides or silver halides.

In a preferred variant of the apparatus, the optical element of the ATR apparatus is covered with a disposable film. This film is additionally mechanically removable and the sample is metered onto the film.

The disposable film is advantageously manufactured from polyethylene.

The optical element of the ATR apparatus is preferably provided with a frame to bound the measurement surface.

The frame may more preferably consist of a ring of hydrophobic material pressed onto the disposable film.

The mechanical ring may be combined with the disposable film or is in particular integrated into the disposable film by embossing techniques.

The apparatus is especially preferably equipped with a digital camera for recording an image of the sample.

The apparatus is optionally additionally equipped with a light source in order to be able to record an image of the sample with the digital camera.

In a particularly preferred variant, the apparatus is additionally equipped with a layer thickness measurement apparatus, especially based on laser backscattering.

The ATR apparatus may comprise an interferometer for performing FTIR spectroscopy.

The readout element of the ATR measurement apparatus appropriately and preferably comprises a Globar and one or more broadband IR detectors which are equipped with dielectric interference filters.

It is also possible to use IR beams of discrete wavelengths from semiconductor components or CO₂ lasers. The semiconductor components consist, for example, of one or more quantum cascade lasers or one or more diode lasers. The lasers are preferably tunable.

In a particularly effective apparatus, the metering apparatus and the optical element of the ATR measurement apparatus are combined on a common substrate.

The optical element of the ATR measurement apparatus may in particular be coupled to the readout unit of the ATR measurement apparatus by means of lightguide elements.

Very particular preference is given to an apparatus in which the metering apparatus, the optical element and the readout element of the ATR measurement apparatus are integrated on a common substrate.

ATR measurement apparatus: An ATR measurement apparatus consists, for example, preferably of an optical element, a readout element and optionally of lightguide elements which connect the optical element to the readout element.

Optical element: The optical element is, for example, an infrared-transparent solid body which has a refractive index higher than that of water or protein layers and is therefore suitable for providing interfaces at which total internal reflection of an incident IR beam takes place. Examples of optical elements are right-angled prisms manufactured from diamond, silicon or germanium, or waveguides of the same materials. Waveguides may either be configured in a planar manner on a substrate, consist of plate waveguides or be waveguides which are configured as optical fibres with a circular cross section.

Readout element: The readout element consists, for example, of one or more IR light sources, optionally optical equipment such as lenses, mirrors, interferometers, wavelength-sensitive filters and one or more IR-sensitive detectors. In general, the chamber in which the IR beam propagates freely can be evacuated in order to eliminate the water absorption.

Lightguide element: A lightguide element is, for example, an appropriately shaped solid body which conducts light by total internal reflection. Examples are IR-conducting optical fibres manufactured from silver halides, chalcogenides and waveguides which are defined photolithographically, for example are configured as silicon microstructures. Lightguide elements may be equipped with lenses in order to couple and decouple the light beams.

The invention is illustrated in detail by way of example with reference to the figures. They show:

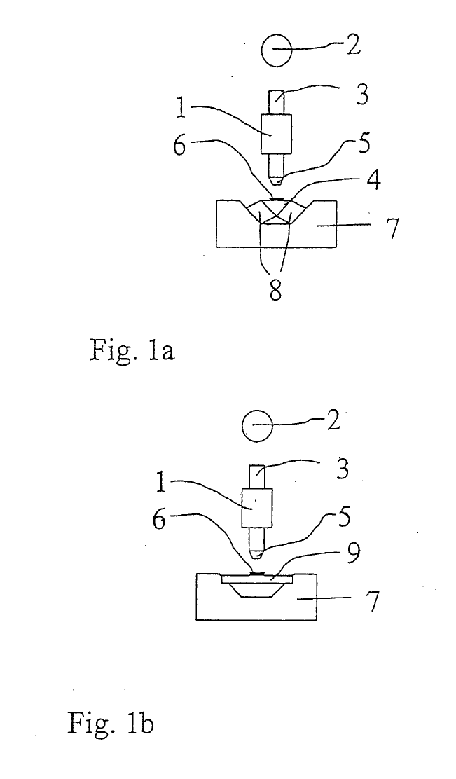

FIG. 1a Metering apparatus and ATR apparatus whose optical element is a prism.

FIG. 1b Metering apparatus and ATR apparatus whose optical element is a plate waveguide.

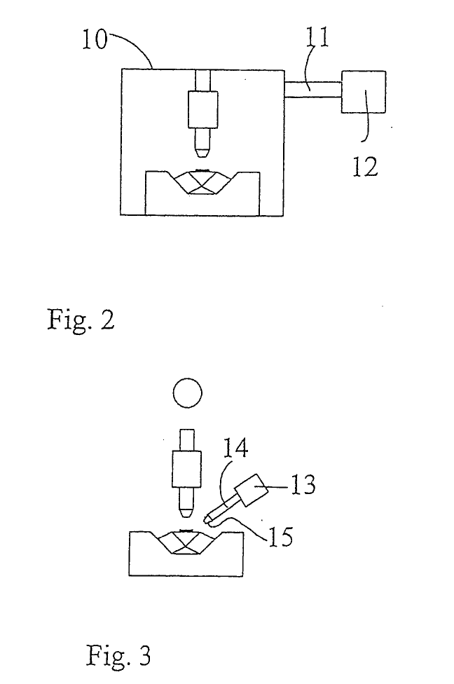

FIG. 2 Apparatus mounted in a casing which can be evacuated.

FIG. 3 Apparatus with additional air ventilator in order to accelerate the drying of the liquid sample.

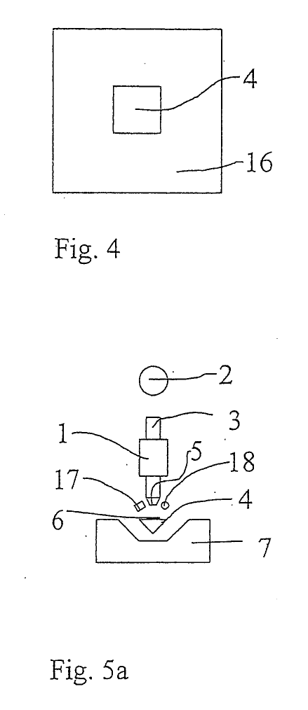

FIG. 4 Plan view of an optical element which is mounted in a holder.

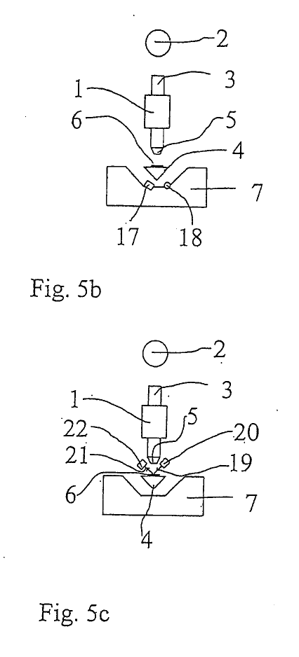

FIG. 5a Apparatus equipped with a unit which is mounted above the optical element for recording a sample image.

FIG. 5b Apparatus equipped with a unit which is mounted below the optical element for recording a sample image.

FIG. 5c Apparatus equipped with a laser backscattering unit which is mounted above the optical element in order to generate location-resolved height information of the sample.

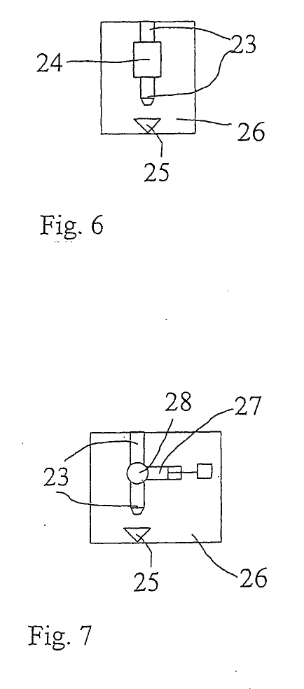

FIG. 6 Metering apparatus and optical element on one chip.

FIG. 7 Syringe-based metering apparatus and optical element on one chip.

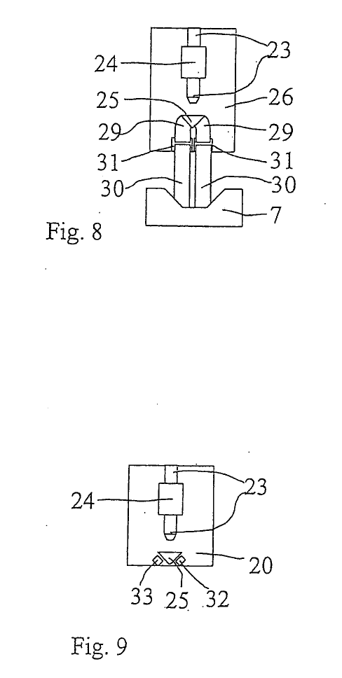

FIG. 8 Metering apparatus and optical element coupled with an evaluation element via planar waveguides and optical fibres on one chip.

FIG. 9 Metering apparatus and ATR apparatus on one chip.

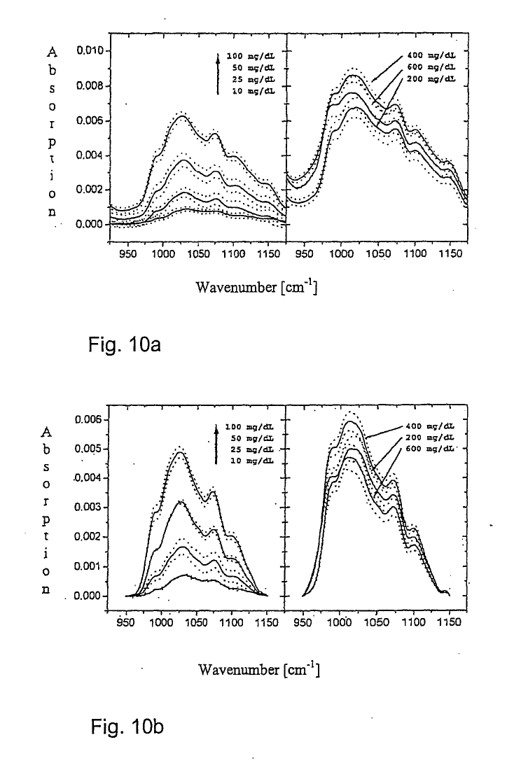

FIG. 10a MIR-ATR spectra of aqueous glucose solutions for different concentrations.

FIG. 10b Baseline-corrected MIR-ATR spectra of aqueous glucose solutions for different concentrations.

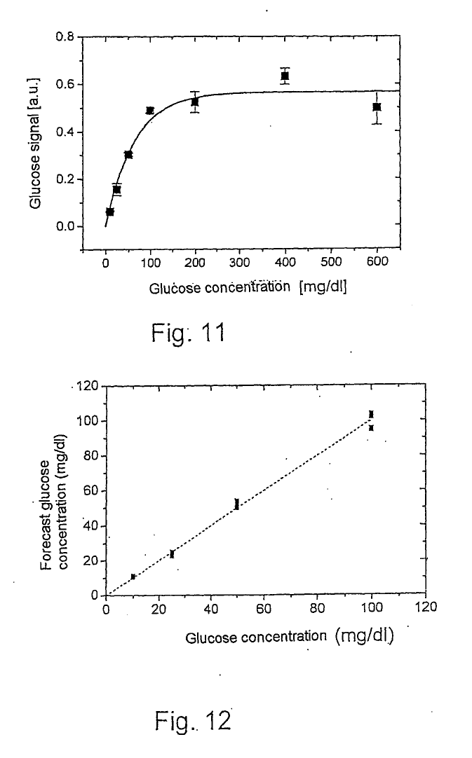

FIG. 11 Glucose signal derived from baseline-corrected MIR-ATR spectra of aqueous glucose solutions for different concentrations.

FIG. 12 Forecast glucose concentrations derived from baseline-corrected MIR-ATR spectra of aqueous glucose solutions for different concentrations in combination with a monoexponential calibration model as a function of the glucose concentration.

The underlying novelty of the invention is the combination of a metering apparatus with an ATR apparatus in order to prepare samples in the sub-μl range of 0.2 nl to 1000 nl in one instrument and to analyse them. These small volumes are advantageous for the analysis of body fluids that can only be obtained in these volumes. For example, for the analysis of interstitial fluid which is obtained through the skin, typical sample volumes are between 100 and 500 nl. The underlying functional principles are explained with reference to FIG. 1a. The metering apparatus 1 aspirates the sample 2 through an inlet line 3 and subsequently meters the sample onto the optical element 4 of the ATR apparatus through an outlet nozzle 5. Here, the optical element is a prism. The subsequent drying of the sample increases the concentration of the analyte and eliminates the water absorption for the MIR spectroscopy. The dry film 6 of the sample is irradiated with an IR beam which is emitted and detected by a readout element 7 and optionally conducted from and to the sample 6 via lightguide elements 8. In the variant of FIG. 1b, the prism is replaced by a planar waveguide 9. The spectroscopic data are analysed in relation to one or more analytes in accordance with the calibration model. This model is derived from the analysis of a test data set by means of PLS partial least squares) or neural networks. The combination of a metering apparatus 1 with an ATR apparatus in one instrument is advantageous, since an ATR apparatus ensures free access to the surface of the optical element 4. No additional means are required, such as reflecting surfaces for diffuse reflection methods or detectors or mirrors or lightguide elements for the transition method, which block the free access to the surface of the sample stage. The sample volume to be analysed is determined by the parameter settings of the metering apparatus 1. Therefore, no cuvettes are required which define the sample volume. Since the ATR method is surface-sensitive as a consequence of the evanescent nature of the IR test beam, the thickness of the metered sample film 6 is not as decisive as in the case of the reflection or transmission method, as long as the layer thickness is distinctly above the penetration depth of the evanescing field.

The sample to be analysed is initially in liquid form. In the context of medical diagnosis, body fluids such as interstitial fluid, blood, serum, plasma, urine, saliva, sweat or lacrimal fluid are preferably investigated. Analytes are all clinically relevant components which can be distinguished by IR spectroscopy: especially glucose, HDL and LDL cholesterol, triglycerides, albumin, total protein content, urea, uric acid, haemoglobin and creatinine. IR spectroscopy is divided into the near infrared region (NIR) between 4000 and 14 000 cm⁻¹ and the middle infrared region between 800 and 4000 cm⁻¹. Both methods can in principle be used for the invention, although MIR spectroscopy is the preferred measurement as a consequence of the higher resolution.

The metering apparatus 1 is a mechanical apparatus which can aspirate and meter the predefined volume of a liquid sample. The underlying function is based on the application of pressure to the sample by means of a piezo crystal or by means of a syringe. Piezo crystal-based metering apparatus are preferably used for ultrasmall volumes between 0.1 and 100 nl, while syringe-based metering apparatus are preferably used for larger volume ranges bordering on this range.

As described above, the sample volume may be metered in a single drop or in a sequence of successive drops. Within the sequence, sufficient time is allowed between the metering of individual drops that the individual drops are dried before the next drop is metered. More than one drop may also be metered per metering step within the sequence.

Sequential metering has the advantage of realising smaller surface areas of the dried film than the method of metering the sample of the same volume in one drop. In order to accelerate the drying process, additional methods may be employed. FIG. 2 shows one example in which the metering apparatus and the ATR measurement apparatus are mounted in a casing 10 which can be evacuated via a line 11 by means of a pump 12.

In another embodiment, illustrated in FIG. 3, the drying is accelerated by a gas stream through the metered sample 6. In addition, the gas stream may be preheated. This may be brought about by an air ventilator 13, a feed line 14 and a nozzle 15. Heat may also be applied by a light source which has significant emission in the IR in order to evaporate the water of the aqueous sample at an accelerated rate. This light source may be a glow lamp which is mounted at the same point as the air nozzle 15 in FIG. 3.

In another embodiment, cf. FIG. 4 (plan view), the optical element 4 is mounted in a holder 16 which is heated, for example, to 40 to 70° C. As a consequence of the thermal bridge between holder 16 and optical element 4, the evaporation of the liquid sample is promoted.

The optical element 4 of the ATR measurement apparatus has at least one surface at which the IR beam is totally reflected. At this interface, the sample film metered onto the optical element 4 is tested by the evanescent field. A suitable surface is a surface of an optical element of the ATR apparatus. Preferred materials for the optical elements are diamond, silicon, zinc selenide or germanium. In another embodiment, optical fibres which consist of chalcogenides or silver halides serve as optical elements. In order to develop the apparatus in the direction of point-of-care applications, the optical element 4 may be equipped with a disposable film, preferably a polymer film, e.g. polyethylene, to protect the surface and for rapid renewal. This film has, for example, a thickness of 100 to 1000 nm and is in close contact, i.e. without any intermediate space, with the optical element. After the spectroscopic measurement, the film is removed mechanically from the optical element 4 and a new film is applied to the optical element.

In order to increase the reproducibility of the ATR signal, the surface area of the sample film on the optical element 4 may be bounded by a frame. The frame may consist of a Teflon ring which is pressed onto the optical element. An alternative consists in applying a hydrophobic ring to the optical element by a hydrophobic stamping process. These rings may also be combined with the abovementioned disposable film, in which case the film spans the base of the Teflon ring or the hydrophobic ring is pressed onto the disposable film. A further alternative consists in the integration of a mechanical ring of the same material as the disposable film into the disposable film by embossing techniques. As an alternative method of determining the surface area of the dry film or even the film shape, an image may be recorded or a laser backscattering scan of the dry film may be carried out. These data afford additional information for the forecasting of analyte concentrations in a calibration. An image of the interface of the total reflection of the optical element is recorded by a CCD camera 17 which is mounted above, cf. FIG. 5a, or below, cf. FIG. 5b, the optical element 4. For the mounting of the camera 17 below the optical element, either a separate light source 18 or the IR radiation itself is utilized as a light source in order to generate an image of the sample film 6. When the camera 17 is mounted above the optical element 4 in a somewhat tilted manner relative to the metering apparatus 1, an additional light source 18 has to be utilized in order to illuminate the sample 6 from above. From the image, the surface area of the sample film 6 is determined by means of commercial image processing software. Moreover, additional information on the topography of the sample film 6 may be derived from a colour image on the basis of optical interference phenomena. These may serve as additional parameters for the quantitative forecasting of analyte concentrations.

In a further embodiment in FIG. 5c, an example of a laser backscattering unit is illustrated. A focused beam 19 is scanned over the sample 6 by a laser 20. Its reflected beam 21 is recorded by a CCD camera 22. Both the laser 20 and the camera 22 may be equipped with, for example, objectives for increasing the resolution. The triangulation principle described affords information for the height information of the sample 6 with respect to the surface of the optical element. Spatial scanning allows the three-dimensional reconstruction of the sample shape. Apart from this set of height information which may be used as additional individual parameters for the forecast of the analyte concentrations, the sample volume may also be determined therefrom and this may be used as an additional parameter instead of the set of the particular height data.

The optical element 4 is integrated into the optical path in order either to carry out Fourier transform infrared spectroscopy (FTIR) or to apply a noninterferometry method. The latter is characterized in that the sample 6 is irradiated with light of discrete wavelengths, wavelength ranges or broadband light (in conjunction with wavelength-sensitive detectors) in the NIR and/or MIR range. Discrete wavelengths may be provided by lasers, for example CO₂ lasers or semiconductor lasers, which may additionally also be tuned. Examples of semiconductor lasers are quantum cascade lasers or diode lasers. Different wavelength ranges are covered by several appropriately different laser sources. Wavelength ranges are provided by broadband light sources, for example a Globar in conjunction with wavelength-sensitive filters. In order to eliminate background light, infrared detectors, for example MCT, DTGS, DLATGS, thermoelement detectors or bolometers are equipped with wavelength-sensitive filters. The filters are based on dielectric multilayers having adjustable wavelength windows. In addition, tunable emitters, filters and detectors which are based, for example, on photonic band gap technology (for example of Ion Optics Inc., Waltham, Mass., USA) may be used. Several detectors which are sensitive to different wavelength or wavelength ranges may be read out successively or simultaneously. The parallel readout has the advantage of eliminating temporal fluctuations of signals.

The combination of a metering apparatus with an ATR apparatus has the potential of being integrated on a common chip. A first step is the integration of the metering apparatus with the optical element 4 on one chip. Channel structures 23 and pressure-generating apparatus 24, for example membrane pumps, or, for example, a prism 25 as an optical element 4 may be generated on a silicon wafer 26 (cf. FIG. 6).

A pressure-generating apparatus may also be realised with piezo crystals which rest on a channel, or by a syringe 27 in conjunction with a three-way cock 28 in FIG. 7 which are both configured in microsystem technology (MEMS). The optical element 4 on the chip in FIG. 6 or FIG. 7 may be connected to the readout element 7 via a lightguide element. These waveguides may be integrated on the chip by means of photolithographic techniques.

Alternatively, they may also consist in separate optical fibres or in a combination of integrated waveguides 29 and optical fibres 30, cf. FIG. 8. The optical fibres may be positioned on the chip by placing them in grooves 31 formed on the chip. The best possible integration is achieved by realising the metering apparatus and the ATR apparatus on a common chip. One embodiment is based on MEMS technology, for example the photonic band gap technology, cf. Ion Optics Inc. In extension of FIG. 6 into FIG. 9, a microbridge 32 is designed as the IR-emitting source and a further microbridge 33 as a detector. Microbridges are, for example, parts of Ion Optics IR gas Sensor Chip™.

Calibration models are realised by mathematical procedures. These models convert data which are derived from spectroscopic data to desired analyte concentrations. The general principle is characterized in that a set of training data is utilized in order to generate a model. This model is subsequently used to determine the concentration for the set of test data. Simple models utilize functions which are based on one or more parameters which generally result from nonlinear fitting processes using the set of training data. A simple example is linear regression analysis. Complex dependencies of the spectroscopic data on the analyte concentrations are modelled by standard partial least squares methods (PLS) or neural networks. These models may even be extended by taking into account several parameters beyond the spectroscopic data, for example morphological parameters such as sample film surface area, sample volume or height profile.

### EXAMPLE

### Method and Apparatus for Detecting Glucose in Aqueous Glucose Solutions

The example describes an embodiment in which an aqueous glucose solution is metered onto an ATR prism by means of a microdropper. This example illustrates the principle of the invention but does not limit the invention thereto.

Aqueous solutions comprising glucose (Fluka, Deisenhofen, Germany, purity>99.5%) in deionized water having concentrations of 10, 25, 50, 100, 200, 400 and 600 mg/dl were prepared. The metering apparatus 1 used was a microdrop AutoDrop system in conjunction with an AD-K-501 metering head and a computer controlled 3D positioning system (all from MicroDrop, Norderstedt, Germany). The tip of the metering head consists of a glass capillary which is shaped to a nozzle (internal diameter=70 μm). From this nozzle, individual drops having a volume of 0.2 nl are expelled by a piezo crystal-based impulse acting on the glass capillary. A method was established that allows the reproducible metering of nanolitre volumes onto the surface of a Golden Gate diamond ATR crystal (Specac, Smyrna, Ga., USA). The glucose solution was dropped on in 20 series, each of which contained two drops, at a rate of 100 Hz. The resulting drops from the first series dried on the prism over 30 s, before the second series was dropped onto the same point. This resulted in a sample amount of 8 nl. The distance between nozzle and prism surface was 0.5 mm. This method was used to obtain dried glucose films having a virtually constant diameter of approx. 200 μm irrespective of the glucose concentration used. The FTIR measurements were carried out with a Bruker Tensor 27 Fourier Transform IR Spectrometer (Bruker GmbH, Ettlingen, Germany). The spectrometer was equipped with a standard Globar as the light source and a DLATGS pyroelectric detector. The Golden Gate ATR system was mounted in the sample chamber of the spectrometer. Reference measurements were recorded with ethanol and water after each sample and cleaning of the ATR crystal surface. This allowed deviation between sample and reference measurements as a consequence of repositioning errors of the ATR crystal in the spectrometer to be prevented. Care was taken that ethanol had fully evaporated before the reference measurement was carried out. Consequently, ambient air served as the reference medium. All spectra were recorded in the spectral region between 500 and 6000 cm⁻¹ with a resolution of 4 cm⁻¹ and averaged 256 times. The time for one measurement was 300 s. Samples were in many cases measured with N=4 to 6. The resulting spectra, cf. FIG. 10a, were subsequently baseline-corrected in the spectral region between 950 and 1150 cm⁻¹, cf. FIG. 10b. Dotted lines in FIG. 10a and FIG. 10b show the variability of the spectra within a standard deviation about the average value within an experimental set of the same glucose concentration. To analyse the signal response function as a function of the concentration c, the area below the baseline-corrected spectrum was defined as the glucose signal s in the region between 950 and 1150 cm⁻¹. FIG. 11 shows a virtually linear response function for the concentration range between 10 and 100 mg/dl. For this range, we have constructed a calibration model which has a monoexponential form s=s₀+A exp (−c/t) with (s₀, A, t) as fitting parameters. The goodness of the model is reflected in a high correlation factor R²=0.997. Consequently, the standard error of the concentration forecast which stems from a leave-one-out cross-validation of 2.6 mg/dl is very small. Forecast glucose concentrations against glucose concentrations are shown in FIG. 12. A forecast concentration value stems from a procedure in which all glucose signal data up to that of the case in question are used to determine the fitting parameters (s₀, A, t). With the aid of these parameters and the left-out glucose signal, the corresponding glucose concentration to be forecast is determined. The fitting routine which is used by the monoexponential model is part of commercially available software (ORIGIN6.1G, OriginLab Corporation, Northampton, Mass., USA). Between 100 and 600 mg/dl, the signal saturates because the sample film thickness exceeds the penetration depth of the evanescent field. However, the non-baseline-corrected spectra from FIG. 10a have sufficient spectrally different information that promises promising use of PLS and neural networks in order to correspondingly extend the forecast range. In summary, the combination described of a metering apparatus with an ATR apparatus demonstrates the sensitivity and reproducibility for the detection of glucose up to 10 mg/dl in an 8 nl sample volume.

1-24. (canceled)

25. A method for analyzing a liquid sample comprising at least one analyte, the method comprising the acts of:
aspirating the sample into a metering apparatus;
metering the sample onto the measurement surface of an optical element of an ATR apparatus via the metering apparatus;
measuring the ATR-IR spectrum; and
analyzing the ATR-IR spectrum with a calibration procedure for concentration determination of the analytes to be detected,
wherein the sample is dried on the measurement surface before the ATR-IR spectrum is measured.

26. The method of claim 25, wherein the sample is interstitial fluid, blood, serum, plasma, urine, saliva, sweat or lacrimal fluid.

27. The method of claim 25, wherein the analyte is glucose, high-density lipoproteins (HDL), low-density lipoproteins (LDL), cholesterol, triglycerides, albumin, total protein, urea, uric acid, hemoglobin, creatinine or combinations thereof.

28. The method of claim 25, wherein the NIR spectrum is measured in the wavenumber range of 800 to 14,000 cm⁻¹.

29. The method of claim 25, wherein the sample has a volume of 0.2 to 1000 nl.

30. The method of claim 25, wherein the sample is applied to the measurement surface of the ATR apparatus as a drop sequence in several acts, each of the drop sequences including at least one drop and the sample being dried between the drop sequences.

31. The method of claim 25, wherein the measurement surface is bounded by a frame on the optical element of the ATR apparatus.

32. The method of claim 25, wherein the metering apparatus is a piezo-driven dropper or a syringe-driven dropper.

33. The method of claim 25, wherein the apparatus is mounted in an evacuable casing, the casing being equipped with an evacuation mechanism.

34. The method of claim 25, wherein the apparatus further includes a blower and a feed line connected thereto, and at least one nozzle that is connected to the feed line and is directed towards the measurement surface of the optical element of the ATR apparatus.

35. The method of claim 25, wherein the optical element of the ATR measurement apparatus is a prism, a planar waveguide or a plate waveguide, the optical element comprising diamond, silicon, zinc selenide or germanium.

36. The method of claim 25, wherein the optical element of the ATR measurement apparatus is an optical fiber manufactured from chalcogenides or silver halides.

37. The method of claim 25, wherein the optical element of the ATR measurement apparatus is covered with a removable disposable film.

38. The method of claim 37, wherein the disposable film consists of polyethylene.

39. The method of claim 25, wherein the optical element of the ATR measurement apparatus is provided with a frame to bound the measurement surface.

40. The method of claim 39, wherein the frame consists of a ring of hydrophobic material pressed onto the disposable film.

41. The method of claim 25, wherein the apparatus further includes a digital camera to record an image of the sample.

42. The method of claim 25, wherein the metering apparatus and the optical element of the ATR measurement apparatus are combined on a common substrate.

43. The method of claim 25, wherein the metering apparatus, the optical element and the readout element of the ATR measurement apparatus are combined on a common substrate.
